(19) United States
(12) Patent Application Publication
(10) Pub. No.: US 2016/0024461 A1
(43) Pub. Date: Jan. 28, 2016

(54) METHOD FOR FABRICATING A CELL-LADEN HYDROGEL CONSTRUCT

(71) Applicant: NATIONAL TAIWAN UNIVERSITY, Taipei City (TW)

(72) Inventors: Jui-Sheng Sun, Taipei City (TW); Pei-Yu Chen, New Taipei City (TW); Hui-Wen Huang, New Taipei City (TW)

(73) Assignee: NATIONAL TAIWAN UNIVERSITY, Taipei City (TW)

(21) Appl. No.: 14/337,853

(22) Filed: Jul. 22, 2014

(51) Int. Cl. C12N 5/00

(52) U.S. Cl. CPC C12N 5/0062

(57) ABSTRACT

The present invention provides a method for fabricating a cell-laden hydrogel construct, comprising the steps of: (a) making a mixture of a physically gelable protein, a first crosslinking enzyme and a cell; (b) extruding the mixture into a second crosslinking enzyme before the mixture is gelled and forming a microgel by physical gelling; and (c) reacting the microgel with the second crosslinking enzyme to fabricate the cell-laden hydrogel construct.

### FIELD OF THE INVENTION

The present invention is related to a method for fabricating a cell-laden macroporous hydrogel construct.

### BACKGROUND OF THE INVENTION

The volume of research in tissue engineering has increased substantially since 1990's. However, only a few products are clinically applicable up to now. These products are either thin or metabolically inactive tissues such as skin, large vessel, trachea, and cartilage. For most tissues or organs to be functional, the constructs should be three-dimensional (3D) and the sizes should be clinically relevant. Creation of such constructs has been limited by many challenges. Commonly used methods of 3D cell culture are cell aggregates, microspheres, in situ forming hydrogels, and preformed porous scaffolds. The aggregates and microspheres lack integral structure. Hydrogels have difficulty in providing sufficient perfusion and mass transfer when the size increased. Although widely used in preparing large construct, preformed porous scaffolds have problems in efficient cell seeding and distribution (Tan, L., Y. Ren, and R. Kuijer, A 1-min method for homogenous cell seeding in porous scaffolds. J Biomater Appl, 2012. 26(7): p. 877-89). The retention of the cells in the scaffold relies on the ability of the cells to adhere on the surface of the scaffold, and less adhesive cells may be lost during perfusion. Furthermore, cells are exposed to non-physiological shear forces. In our bodies, only endothelial cells are subject to blood flow, and the exchange of nutrition and waste is through perfused capillary systems. Cells laden in hydrogels can be protected from the hydrodynamic forces if they are perfused over the surfaces.

A critical obstacle in tissue engineering is the inability to maintain large masses of living cells upon transfer from in vitro culture conditions into the host in vivo (Ko, H. C., B. K. Milthorpe, and C. D. McFarland, Engineering thick tissues—the vascularisation problem. Eur Cell Mater, 2007. 14: p. 1-18; discussion 18-9; Kaully, T., et al., Vascularization—the conduit to viable engineered tissues. Tissue Eng Part B, 2009. 15(2): p. 159-169). In vitro tissue cultures may simply depend on the perfusion of culture media. However, for large 3D engineered tissue to survive after implantation, a well established capillary microcirculation is necessary to maintain the viability and function of the tissue constructs (Novosel, E. C., C. Kleinhans, and P. J. Kluger, Vascularization is the key challenge in tissue engineering. Adv Drug Deliv Rev, 2011. 63(4-5): p. 300-11). It has been reported that constructs seeded with endothelial cells and mesenchymal supportive cells or that contained rudimentary vascular structures may accelerate the inoculation with host vessels to form mature and perfused vascular networks. Nevertheless, these studies involved small tissue constructs. When the size of engineered tissue increased, a delay in establishing the blood circulation may cause necrosis inside the tissue. Efforts have been attempted to create vascular analogues in vitro, with partial achievement in the in vitro formation of multi-branching networks of cells, and even lumens. However, there is still no report of tissue constructs incorporated with stable patent vascular network in vitro. To achieve this task, it is necessary to integrate all the factors together. Beside the endothelial cells, supportive cells, and growth factors, another key component is a stably perfused scaffold with suitable attaching surface for endothelial cells.

Efforts have been made to combine the advantages of porous scaffolds and hydrogels by designing methods to produce large porous hydrogel scaffolds, which are able to encapsulate viable cells in situ, while at the same time can provide plentiful porosity for adequate perfusion. Photolithography was used to fabricate cell-encapsulated Poly(ethylene glycol)diacrylate (PEGDA) microgels, which were further successfully assembled into porous structures by another photopolymerization or a polypeptide cross-linker (Du, Y., et al., Directed assembly of cell-laden microgels for fabrication of 3D tissue constructs. Proc Natl Acad Sci USA, 2008. 105(28): p. 9522-7; Liu, B., et al., Modularly assembled porous cell-laden hydrogels. Biomaterials, 2010. 31(18): p. 4918-25). However, the risk of damage to DNA by UV light is a concern (de Gruijl, F. R., H. J. van Kranen, and L. H. Mullenders, UV-induced DNA damage, repair mutations and oncogenic pathways in skin cancer. J Photochem Photobiol B, 2001. 63(1-3): p. 19-27; Sinha, R. P. and D. P. Hader, UV-induced DNA damage and repair: a review. Photochem Photobiol Sci, 2002. 1(4): p. 225-36). Furthermore, poor biodegradability of the material and lack of biological cues may set some limitations in clinical applications.

A microbial transglutaminase (mTGase) derived from Streptoverticillium species has been used widely in food processing. It can change the food texture by catalyzing the formation of peptide bonds between the γ-carboxyamide group of glutamine and the ε-amino group of lysine. Enzymatic cross-linking of gelatin-based materials using this mTGase has been investigated for various applications such as drug delivery, injectable cell delivery, and scaffold fabrication (Fuchs, S., et al., Transglutaminase: new insights into gelatin nanoparticle cross-linking. J Microencapsul, 2010. 27(8): p. 747-54; Kuwahara, K., et al., Cell delivery using an injectable and adhesive transglutaminase-gelatin gel. Tissue Eng Part C Methods, 2010. 16(4): p. 609-18; Yung, C. W., et al., Transglutaminase crosslinked gelatin as a tissue engineering scaffold. J Biomed Mater Res A, 2007. 83(4): p. 1039-46). Cells have been reported to tolerate the cross-linking process well, and the activity of the exogenous mTGase may be terminated by cell-secreted proteinases.

It is believed that cell-laden perfusable macro-porous hydrogel scaffolds is one viable solution to fabricating tissue grafts of clinically relevant size because they could be scaled up without compromise of their efficient cell-seeding, integral structure, oxygen delivery, and mass transfer. Beside the target tissue engineered in the hydrogel, stable endothelium-covered microvasculature might be engineered on the stable hydrogel surface in the interconnected pores. PEGDA and photolithography were not employed due to their inherent disadvantages. Instead, gelatin is chosen as the principle matrix and mTGase is chosen as the cross-linking agent. Several methods of scaffold fabrication were conceived and tested. The present invention goes through fabrication of the scaffold, characterization of cell growth in the scaffold, and modeling of pre-vascularized tissue graft with bone tissue engineering.

For tissue engineering, scaffolds possessing interconnected pores have been identified as a requirement (Hutmacher, D. W., Scaffold design and fabrication technologies for engineering tissues—state of the art and future perspectives. Journal of Biomaterials Science, Polymer Edition, 2001. 12(1): p. 107-124). Several methods have been developed to process synthetic and natural scaffold materials into porous structures, including conventional solvent-casting particulate leaching, gas foaming, phase separation, freeze drying, melt moulding, fiber bonding, electrospinning etc., and computer-aided solid freeform fabrication methods such as 3D printing, fused deposition modeling, stereolithography. Unlike that for preformed scaffolds, the methods employed for the fabrication of cell-laden hydrogels must be completed in mild conditions tolerable for the cells. Modifications are needed before these methods could be applied in the fabrication of cell-laden hydrogels. Most of the conventional methods are to create pores in a construct, while the fiber bonding, electrospinning, and solid freeform fabrication methods are to build small parts into an integral construct. Both approaches should be modified to adapt to the hydrogel material and in the existence of cells. The porogen must be able to be removed in aqueous solution, or, a secondary cross-linking of the hydrogel built parts could be done.

Hydrogels are formed by crosslinking polymer chains—through physical, ionic or covalent interactions—and are well known for their ability to absorb water. Hydrogels possess a good biocompatibility in general. Their three-dimensional (3D) structure is excellent to mimic extracellular environments and, consequently, they are frequently used to encapsulate cells in a 3D-microenvironment (Teixeira, L. S., et al., Enzyme-catalyzed crosslinkable hydrogels: emerging strategies for tissue engineering. Biomaterials, 2012. 33(5): p. 1281-90.)

In situ gelling methods suitable for delivering hydrogels with drugs, biomolecules, or encapsulated cells by injection were of particular interest. A polymer solution can be prepared and allowed to gel in situ, after photopolymerization, chemical crosslinking, enzymatic crosslinking, ionic crosslinking, or in response to an environmental stimulus such as temperature, pH or ionic strength of the surrounding medium. Crosslinks have to be present in a hydrogel in order to prevent dissolution of the hydrophilic polymer chains in an aqueous environment. In chemically crosslinked gels, covalent bonds are present between different polymer chains. In physically gelled gels, dissolution is prevented by physical interactions, which exist between different polymer chains.

There are several advantageous characteristics of enzymatic crosslinking of the biomaterials making it one of the preferable methods for in situ gelation to encapsulate viable cells. Most of the enzymes are involved in biological functions in nature and catalyze the reactions at body temperature, neutral pH, and aqueous environment. With the formation of covalent bonds, the enzymatically crosslinked hydrogels are stable and can resist subsequent changes of in vitro and in vivo environment. Due to the site specificity of enzyme, unwanted side reactions or toxicity that can occur with photo-initiator or organic solvents could be avoided, and functional groups in the biomaterials are mostly uncompromised. Barbetta et al. compared gelatin scaffolds that had been obtained by radical polymerization with those obtained by mTGase crosslinking They found that the enzymatically crosslinked scaffold showed reduced cytotoxicity and, furthermore, that hepatocytes that were cultured on these scaffolds exhibited a more differentiated phenotype, as demonstrated by the expression and correct localization of key adhesion proteins (Barbetta, A., et al., Enzymatic cross-linking versus radical polymerization in the preparation of gelatin PolyHIPEs and their performance as scaffolds in the culture of hepatocytes. Biomacromolecules, 2006. 7(11): p. 3059-68). The enzyme-mediated site-specific coupling of ligands allowed extensive cell spreading, proliferation and migration, as well as proteolytic matrix degradation by cell-derived matrix metalloproteinase's (MMPs).

Physical gelling methods are also important in the fabrication of cell-laden hydrogel. Cell-tolerable conditions can be designed. Most importantly, the processes are usually fast allowing prompt formation and easy control of desired shape of the hydrogels. However, physical gelling is reversible, and the hydrogels will lose their strength and shape. The utilization of physical gelling as well as enzymatical cross-linking in the process can provide the best chance to fabricate stable, long-lasting hydrogel constructs rapidly and accurately.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows the structure of macro-porous gelatin hydrogel constructs. SEM shows curled and gathered filaments forming an integral construct with interstices among the filaments (A). Confocal microscopy shows that the full length (except the limited segments bond to the adjacent filaments) of every single hydrogel filament is in direct contact with the medium; Red: tetramethylrhodamine-dextran, labeling the hydrogel filaments; Green: fluorescein-dextran, dissolved in medium, delineating the spaces outside the hydrogel filaments (B). C represents 3D image of confocal microscopy, showing interconnected medium-occupied spaces (green).

FIG. 2 shows the porosity and permeability of constructs fabricated with different mesh size and gel volume fraction. (A) Porosity: Less gel volume fraction results in larger porosity. The differences between constructs of different filament thickness are not significant. (B) Permeability: Less gel volume fraction and larger filament thickness results in higher permeability.

FIG. 3 shows the viability and proliferation of encapsulated NIH 3T3 Cells (1×10⁶/mL hydrogel) under static and perfusion culture of the constructs. A shows that the percentage of survival cells is constantly larger than 90%. The differences between static culture and perfusion culture are significant, p<0.01. B shows that cell proliferation is evaluated by total cell count in a construct. The differences between static culture and perfusion culture at day 4 and day 7 are significant, p<0.05 and 0.01 respectively.

FIG. 4 shows the morphology and growth pattern of different cell types cultured in the constructs. (A) to (C) show the confocal microscopy of NIH 3T3 cells in the constructs at day 0, 4, and 7 under perfusion culture. Upper-left: Eth-1, staining dead cells; upper-right: calcein AM, staining live cells; lower-left: Hoechst 33342, staining cell nuclei; lower-right: merged images. (D) to (F) show the epifluorescence microscopy (stained with calcein AM) and SEM of hADSCs after 7 days of culture under perfusion. Human ADSCs migrates out of the hydrogel, and attaches on the surface of the filaments. Abundant ECM is observed. (G) shows the confocal microscopy of hADSCs, 3 weeks after perfusion culture in adipogenic medium since day 0. Differentiated adipocytes reside in the hydrogel. Upper-left: BODIPY 493/503, staining neutral lipid droplets; upper-right: light field image; lower-left: Hoechst 33342, staining nuclei; lower-right: merged image. (H) shows the light field microscopy of HuH-7 cells. Initially, single cell suspension is used for encapsulation. After 7 days of perfusion culture, multicellular spheres are noted inside the hydrogel. (I) and (J) show confocal microscopy of HuH-7 cells 7 days after perfusion and static culture respectively. The size of cell spheres is obvious larger under perfusion culture than under static culture. Left: calcein AM, staining live cells; middle: Eth-1, staining dead cells; right: Hoechst 33342, staining cell nuclei. (K) and (L) show 3D images of confocal microscopy and SEM after 3 days of static culture and 2 weeks of perfusion culture. HUVECs are seeded in the constructs with hADSCs encapsulated inside the hydrogel. HUVECs covers the surface evenly. Green: CD31. Red: α-smooth muscle actin. Blue: Hoechst 33342, nuclei.

FIG. 5 shows the gross appearance of osteogenic induction of hADSC. Constructs with perfustion culture (lower row) shows more obvious opacification than that in static culture.

FIG. 6 shows the fluorescence microscopy of osteogenic induction of hADSC in 2D culture and in gelatin hydrogel 3D cultures. Mineralization nodules are observed after 7 days (A) or 14 days (B) of osteogenic induction. 3D cultures show more mineralization nodules than 2D culture, and perfusion culture shows even more vigorous mineralization. Upper: light field. Lower: fluorescence of incorporated tetracycline.

FIG. 7 shows the Semiquantitative calcium assay of the bone tissue constructs. The perfused constructs show significantly more calcium formation than static cultured constructs.

FIG. 8 shows that engineered bone graft subsequently seeded with HUVECs after 7 days of induction yields even and complete covering of the pore surface. Blue: Hoechst 33342, nuclei; Red: Xylenol Orange, mineral stain; Green: CD31, endothelial cells.

### SUMMARY OF THE INVENTION

The present invention provides a method for fabricating a cell-laden hydrogel construct, comprising the steps of: (a) making a mixture of a physically gelable protein, a first crosslinking enzyme and a cell; (b) extruding the mixture into a second crosslinking enzyme before the mixture is gelled and forming a microgel by physical gelling; and (c) reacting the microgel with the second crosslinking enzyme to fabricate the cell-laden hydrogel construct.

### DETAIL DESCRIPTION OF THE INVENTION

Unless otherwise specified, “a” or “an” means “one or more”.

### Microbial Transglutaminase

Transglutaminases are a family of enzymes that catalyze the formation of a peptide bond between an amine group and the γ-carboxamide group of protein-bound or peptide-bound glutamine. Bonds formed by transglutaminase exhibit a high resistance to proteolytic degradation. Transglutaminases are present in most animal tissues and body fluids, and are involved in several biological processes, including blood clotting, wound healing, epidermal keratinization, and stiffening of the erythrocyte membrane.

Microbial transglutaminase (mTGase) was first reported produced by Streptoverticillium mobaraense. It is now commonly used for food processing to improve food flavor, appearance and texture.

The eukaryotic transglutaminases are calcium dependent, but transglutaminases from microbial origin are not. Besides, its high level of activity over a wide range of temperature and pH make the enzyme amenable to a wide variety of gel-formation techniques and conditions in short time. McDermott et al. in a study of biomimetic tissue adhesive showed that the gelling can be accomplished within minutes (McDermott, M. K., et al., Mechanical properties of biomimetic tissue adhesive based on the microbial transglutaminase-catalyzed crosslinking of gelatin. Biomacromolecules, 2004. 5(4): p. 1270-9).

The potential of mTGase in catalyzing the crosslink of biological materials, mainly collagen- or gelatin-based, for tissue engineering has been reported by groups since 2005. Broderick et al. characterized the mechanical properties of the crosslinked gelatins and confirmed their cytocompatibility by culturing NIH 3T3 fibroblast on the gelatin gel (Broderick, E. P., et al., Enzymatic stabilization of gelatin-based scaffolds. J Biomed Mater Res B Appl Biomater, 2005. 72(1): p. 37-42). Yung et al. used encapsulated HEK293 cells to show the biocompatibility of mTG-crosslinked gelatin hydrogels (Yung, C. W., et al., Transglutaminase crosslinked gelatin as a tissue engineering scaffold. J Biomed Mater Res A, 2007. 83(4): p. 1039-46). Garcia et al. used bovine collagen from calfskin crosslinked with mTGase to fabricate dermal graft and found it suitable for cell migration, preventing wound contraction, stimulating epithelialization, and neoangiogenesis without inducing any significant inflammatory reaction (Garcia, Y., et al., Towards development of a dermal rudiment for enhanced wound healing response. Biomaterials, 2008. 29(7): p. 857-68).

Beside the applications in fabricating preformed porous scaffold or hydrogel scaffold, mTGase has also been investigated of its use in injectable cell carrier, drug delivery system and microfluidic device.

### Gelatin

Gelatin is a mix of peptides and proteins derived from hydrolysis of collagen, and is commonly used for medical and pharmaceutical applications. It is biodegradable and has good biocompatibility. It also has plenty of function groups suitable for modifications and has a lot of adhesion sequences recognized by cells. As a result, it has been widely investigated for the applications of drug delivery and tissue engineering.

Gelatin can be positively charged or negatively charged in physiological conditions. Two different types of gelatin can be produced depending on the method of pretreatment prior to the extraction process. The type A gelatin (alkaline gelatin) is that received acidic pretreatment usually used in porcine skin. The type B gelatin (acidic gelatin) is that with alkaline pretreatment used for bovine hides with more complex collagen. The alkaline process targets the amide groups of asparagine and glutamine, hydrolyses them into carboxyl groups, and converts many of these residues to aspartate and glutamate. The alkaline processed gelatin possesses a greater proportion of carboxyl groups, rendering it negatively charged and lowering its isoelectric point (IEP). In contrast, acidic pre-treatment does little to affect the amide groups, and has an IEP similar to collagen. The flexibility in IEP make gelatin versatile as a carrier for drugs or biomolecules with different charges.

In addition to gelling by chemical crosslinking, gelatin can undergo physical gelling with the change of temperature. Gelatin has a upper critical solution temperature (UCST), and cooling leads to reversible gelling (Yung, C. W., et al., Transglutaminase crosslinked gelatin as a tissue engineering scaffold. J Biomed Mater Res A, 2007. 83(4): p. 1039-46).

Large and perfusable scaffolds are prerequisite for tissue engineering to be applicable in most clinical conditions. However, when the size increased, preformed porous scaffolds have problems in cell seeding and distribution; and, on the other hand, cell-laden hydrogels are unable to maintain sufficient oxygen delivery and mass transfer. A method to fabricate macro-porous constructs of hydrogel in cell-tolerable processes provides a solution to overcome the deficiencies of conventional cell-laden hydrogels or preformed porous scaffolds.

In the present invention, it is disclosed a very simple method to fabricate large, perfusable, macro-porous, and cell-laden hydrogel constructs. The first step is to get cell-laden gelatin hydrogel filaments, and the second step is to let the filaments bind into integral constructs. The entire process makes use of only common, inexpensive reagents and labwares. This method is a workhorse for 3D cell cultures and tissue engineering.

The combination of gelatin as the matrix and mTGase as the cross-linking agent possesses several advantages. Beside its good biocompatibility and biodegradability, gelatin, as the partially hydrolysed product of collagen, has plenty of adhesion sequences and functional groups. Unlike chemical cross-linking, enzymatic cross-linking is site-specific, leaving other functional groups unaltered. Cells tolerated the mTGase and the fabrication process well. During the 7-day culture period, biological functions of cells including adhesion, migration, and proliferation could be observed in the constructs. Interestingly, adhesive cells such as the NIH 3T3 cells showed out-stretched morphology and actively migrated to the outer surface of the gelatin filaments; while on the other hand, the HuH-7 cells retained inside the gelatin filament in the form of cell spheres. The mTGase mediated cross-linking is peptide bond, and the acellular constructs are stable for more than one month. Cells in the constructs can actively modify the gelatin hydrogel, not only degradation but also new matrix formation. Shrinkage of the size did occur, especially during the first two days, but the overall shape and handling property didn't change remarkably after 7 days of culture.

The constructs showed good porosity and permeability, which could be controlled by altering the thickness or the volume fraction of the gelatin filaments. The fluorophore-labeled fluid could fill all the spaces and outline every gelatin filament well as shown by confocal microscopy, indicating that perfusion of the constructs can deliver culture media everywhere inside them. The measured permeability of the constructs was about 10⁻¹⁰-10⁻¹¹ m², which was 1000× higher than that in the report of Hwang et al. (Nakamura, M., et al., Biomatrices and biomaterials for future developments of bioprinting and biofabrication. Biofabrication, 2010. 2(1): p. 014110). Perfusion of the constructs did have observable effects on the cultured cells. Cell numbers were significantly higher in the perfused group than in the static culture group, suggesting effective perfusion and mass transfer. With the ability to fabricate gelatin filaments as thin as 200 μm, the distance for oxygen diffusion would be no farther than 100 μm, usually adequate for cell survival. Perfusion of up to 6 cm³ cylinder constructs in regular 10-mL syringes has been demonstrated in this invention, and the cells and constructs tolerated it well. The size could be even larger and the shape of the constructs could be changed easily by changing the mold, although a designed perfusion system may be required to ensure proper flow kinematics.

The stably perfused constructs fabricated by the present method provides a useful model for prevascularization. In the embodiment of bone tissue graft, HUVECs were able to attach on and cover the surface of the gelatin filaments well, with engineered target tissue inside the hydrogel construct.

The present invention adopted the approach of vasculogenesis by providing cells to assemble first and expecting the remodeling of the vascular network during extended period of perfusion or after implanted in vivo. It can also be used more like angiogenesis by seeding microvessel fragments instead of cells to allow sprouting and invading into the construct. For both approaches, and even more preferable in combination, to be workable in vitro, a stably perfused system is required which is exactly what we attempted to establish in the present invention.

The present method of fabrication is of great versatility, and can be utilized in various ways. One can mix different types of cells into the gelatin filaments to allow self-rearrangement in the culture. Otherwise, different types of cells can be embedded in separate gels and then assembled together. Moreover, the gelatin matrix is known to be able to adopt modifications of additives for desired biological cues or mechanical properties. For even more delicate engineering, gelatin is a suitable material for bioprinting if the physical and chemical characteristics are handled properly. For example, physical gelation by controlling the temperature can help to set the structure and shape rapidly; and enzymatic cross-linking with mTGase can further stabilize the construct with covalent bonds.

Tissue engineering is the inception of idea and therefore the primary application of the present method. Bone tissue engineering further benefits from incorporation of osteo-inductive materials, growth factors, and by mechanical stimulations; osteochondral constructs is realized by biphasic organization containing different cells and growth factors. One of the most important strength of the hydrogel embedding scaffold of the present invention is the ability to support the real 3D cell-to-cell relationship, not 2D adhesion culture in 3D scaffold. With single-cell encapsulation, cells undergo migration and rearrangement. For the types of cells whose vitality is dependent on cell contact, cell aggregates is encapsulated instead. Another approach is to encapsulate tissue digestion fragments with the primary structures preserved. It is important for preserving special functions of highly organized tissue like endocrine glands.

Therefore, the present invention provides a method for fabricating a cell-laden hydrogel construct, comprising the steps of: (a) making a mixture of a physically gelable protein, a first crosslinking enzyme and a cell; (b) extruding the mixture into a second crosslinking enzyme before the mixture is gelled and forming a microgel by physical gelling; and (c) reacting the microgel with the second crosslinking enzyme to fabricate the cell-laden hydrogel construct.

In the preferred embodiment of the present invention, the concentration of the physically gelable protein in the mixture is from 24% to 1% w/v. In the more preferred embodiment of the present invention, the concentration of the physically gelable protein in the mixture is from 18% to 1.5% w/v. In another more preferred embodiment of the present invention, the concentration of the physically gelable protein in the mixture is from 12% to 3% w/v.

In the preferred embodiment of the present invention, the concentration of the first crosslinking enzyme in the mixture is from 0.6% to 0.01% w/v. In the more preferred embodiment of the present invention, the concentration of the first crosslinking enzyme in the mixture is from 0.45% to 0.25% w/v. In another more preferred embodiment of the present invention, the concentration of the first crosslinking enzyme in the mixture is from 0.3% to 0.05% w/v.

In the preferred embodiment of the present invention, the temperature of the second crosslinking enzyme is from 30° C. to 0° C. and the concentration of the second crosslinking enzyme is from 0.6% to 0.01% w/v. In the more preferred embodiment of the present invention, the concentration of the second crosslinking enzyme is from 0.45% to 0.25% w/v. In another more preferred embodiment of the present invention, the concentration of the second crosslinking enzyme is from 0.3% to 0.05% w/v.

Based on the present invention, the diameter of the microgel is from 400 μm to 100 μm. In the preferred embodiment of the present invention, the physically gelable protein is a protein that has physical gelling property below body temperature including collagen-derived proteins. In the more preferred embodiment of the present invention, the physically gelable protein is gelatin or gelatin-based mixture.

In the preferred embodiment of the present invention, the first or second crosslinking enzyme is transglutaminase, tyrosinase, peroxidases, kinases, phosphotases, thermolysin, or oxidases. In the more preferred embodiment of the present invention, the transglutaminase is tissue transglutaminases, recombinant human transglutaminases or microbial transglutaminase. In another more preferred embodiment of the present invention, the transglutaminase is microbial transglutaminase.

Based on the present invention, the extruding is through a syringe having a needle. In the preferred embodiment of the present invention, the cell is a stem cell, a 3T3 or other immortalized cell line, a HuH-7 or other tumor cell line, a human primary chondrocyte, a mouse primary hepatocyte or other primary cell. In the more preferred embodiment of the present invention, the stem cell is a mesenchymal stem cell, or an adipose-derived stem cell (ADSC).

Compare to the enzymatically gelling, physical gelling by directly extruding the mixture into the solution that is lower than the body temperature has the advantage of: (1) no need to wait for the mixture to gel first; (2) the cell has higher viability; (3) it is easier to control the volume of the cell-laden hydrogel construct; (4) every microgel forms evenly through the syringe having a needle and (5) the arrangement of the microgel is precisely controlled. Based on the present invention, the method is used for 3D bioprinter, 3D cell culture, drug testing platform, extracorporeal life supporting system or tissue engineering.

### EXAMPLES

The examples below are non-limiting and are merely representative of various aspects and features of the present invention.

### Preparation of Gelatin Solution, mTGase Solution

Gelatin solution with w/v concentration 12%, 10%, 8%, 6%, and 4% were prepared for experiments. (An 8% solution was used in the experiments not otherwise stated.) To prepare the 12% w/v gelatin solution, 6 g of gelatin (type A, 175 Bloom; Sigma-Aldrich, St. Louis, Mo.) was dissolved in 40 mL of complete medium (Dulbecco's Modified Eagle Medium (DMEM, Life Technologies, Gaithersburg, Md.) with 10% fetal bovine serum (FBS; Life Technologies) and 100 U/mL penicillin-streptomycin (Sigma-Aldrich)) in 50° C. water bath for 1 hour; after adjusted to pH 7.4, complete medium was added to reach the final volume of 50 mL, and the solution was sterilized by passing through a 0.22 μm filter while kept at 37° C. Sterile 12% gelatin solution was diluted with appropriate volume of complete medium to obtain other concentration.

### Fabrication of Macro-Porous Gelatin Hydrogel Constructs

An 8:1:1 mixture of gelatin, 0.1% w/v mTGase, and cell suspension reacted to form the hydrogel. Cells were suspended in complete medium at 1×10⁷/mL. The final concentration of the mixture would be 9.6%, 8.0%, 6.4%, 4.8%, and 3.2% w/v of gelatin, 0.01% mTGase, and 1×10⁶/mL cells. For experiments without cells, complete medium was added instead of cell suspension.

For physical gelling, one hundred μL of gelatin mixture (8:1:1 mixture of gelatin, 0.1% w/v mTGase, and cell suspension) was drawn into a 1-mL syringe, and a 30 G ¼ needle (160-μm ID) was attached on the nozzle. The ungelled gelatin mixture was then extruded through the needle into 150 μL of ice-cold 0.03% mTGase in DMEM to form filament suspension in a 96-well plate. After 30 minutes of incubation in room temperature, a 6.4-mm OD×7.8 mm hydrogel construct was obtained. It could be transferred to a 6-well plate for static culture or to a 10-mL syringe for perfusion.

### Cell Culture

NIH 3T3 cells were purchased from Bioresource Collection and Research Center (60008, BCRC, Hsinchu, Taiwan), and cultured in DMEM supplemented with 10% FBS and 100 U/mL penicillin-streptomycin.

Human adipose-derived stem cells (hADSCs) were purchased from Life Technologies, and maintained with MesenPro RS Basal Medium with MesenPro RS Growth Supplement (Life Technologies), 2 mM L-glutamine (Life Technologies) and 100 U/mL penicillin-streptomycin.

Human hepatoma HuH-7 cells were generously provided by Dr. Shang-Yi Huang and routinely cultured in DMEM supplemented with 10% FBS and 100 U/mL penicillin-streptomycin.

HUVECs were purchased from BCRC (H-UV001), and cultured in 90% Medium 199 (Sigma-Aldrich) with 25 U/ml heparin (Sigma-Aldrich), 30 μg/ml endothelial cell growth supplement (ECGS; Millipore, Billerica, Mass.), 10% FBS and 100 U/mL Penicillin-Streptomycin. For the experiment of endothelial cell growth, P3 to P5 cells were used. Constructs containing hADSCs about 6 mL in size were fabricated as described, using 10-mL syringes as the molds. Seeding of the HUVECs was accomplished by using the plunger to squeeze out medium, and then draw in 3 mL of cell suspension with 1×10⁶/mL HUVECs. After incubated for 1 hour, the constructs were replenished with medium to the 10-mL mark. The constructs were statically cultured for 3 days, and then cultured under perfusion condition.

### Epifluorescence Microscopy

Pieces of hydrogel were sampled from the constructs and spread on the bottom of a 24-well plate. After staining, the samples were inspected on a Nikon TS100F inverted epifluorescence microscope (Nikon, Tokyo, Japan).

### Confocal Microscopy

For minimizing distortion of the constructs while maintaining high-quality imaging using an upright confocal microscope, slides were modified to accommodate thick samples. Rubber rings 1.5 mm in thickness were attached to the slides with cyanoacrylate, forming flat-bottom wells. Samples were cut into 1.5 mm slices, stained, and transferred into the wells. The samples were immersed in PBS and cover glasses were applied on the wells. The slides were imaged using a Leica TCS SP5 confocal microscope (Leica, Wetzlar, Germany) or a Zeiss LSM 780 confocal microscope (Carl Zeiss, Jena, Germany).

### Scanning Electron Microscopy (SEM)

Ten percent formalin/PBS-fixed constructs with or without cells were dehydrated by treatment with a series of graded ethanol solutions and followed by critical point drying with HCP-2 (Hitachi, Tokyo, Japan). They were subsequently coated with gold ions by Ion Sputter (E101; Hitachi) and then imaged by Hitachi S-4800 Field Emission Scanning Electron Microscope (Hitachi).

### Example 1

### The Gross Properties of the Macro-Porous Gelatin Construct

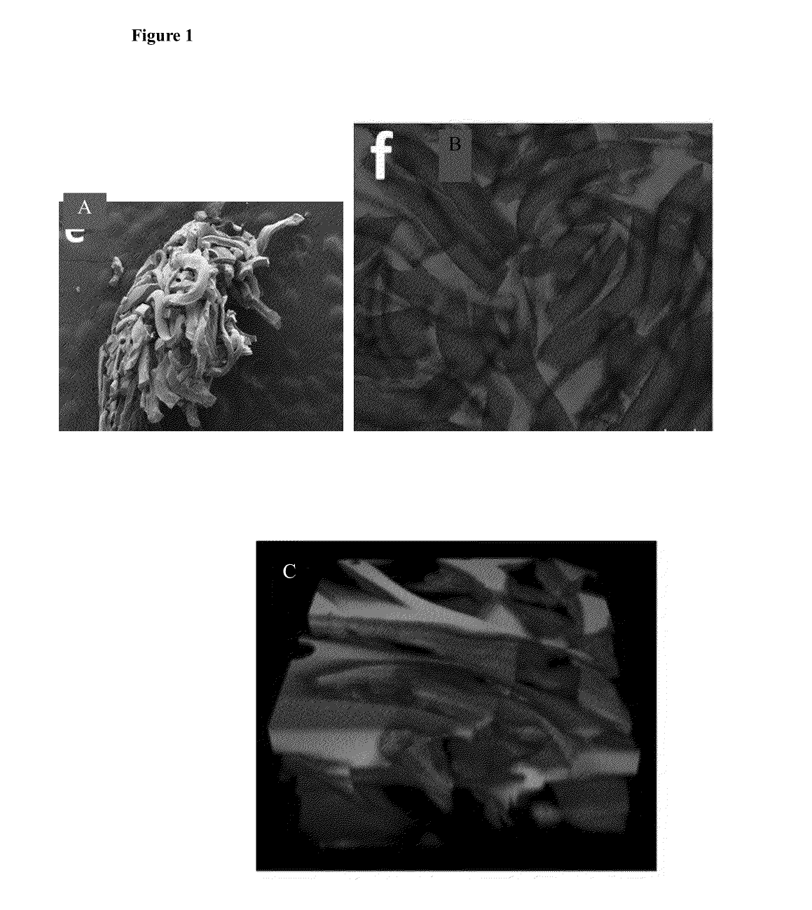

The size and shape were determined by the molds. The constructs looked like sponge, and absorb aqueous fluid. They could be squeezed and then expand spontaneously after pressure released. Under SEM, the filaments curled up and gathered into a cake, with interconnected interstices among them (FIG. 1A). By using tetramethylrhodamine-dextran and fluorescein-dextran to label the filaments and the medium respectively, confocal microscopy demonstrated that every single hydrogel filament was outlined by the medium without a void (FIG. 1B). Three-dimensional image showed that the medium-occupied interstices were well interconnected, allowing perfused medium to flow around all the filaments (FIG. 1C).

### Example 2

### Porosity and Permeability of the Constructs

The structure and macro-porosity of the constructs was evaluated by tetramethylrhodamine-dextran and fluorescein-dextran (MW 2,000 kDa, 0.02 mM; Life Technologies), labeling the hydrogel filaments and the interstices filled with medium respectively. Six constructs of each group were examined under confocal microscopy, and nine regions of a construct were sampled systematically. Images were analyzed by ImageJ software V1.45 s (National Institutes of Health, Bethesda, Md.) to measure the percentage of fluorescein-labeled area.

The permeability of each hydrogel construct to phosphate-buffered saline (PBS) can be characterized according to Darcy's Law, by measuring the rate of flow through the constructs under a known hydrostatic pressure (Swartz, M. A. and M. E. Fleury, Interstitial flow and its effects in soft tissues. Annu Rev Biomed Eng, 2007. 9: p. 229-56; Carr, M. E., Jr., L. L. Shen, and J. Hermans, Mass-length ratio of fibrin fibers from gel permeation and light scattering. Biopolymers, 1977. 16(1): p. 1-15). The permeability constant K=qLμ/pA, where q is the volumetric flow rate, L is the length of the sample in the direction of flow, μ is the fluid viscosity (9.3×10⁻⁴ Pa s for PBS), p is the pressure drop across the specimen, and A is the cross-sectional area of the sample.

Hydrogels were fabricated into 6-mL cylindrical constructs in the barrel of 10-mL syringes as described. Using the setting adapted from that of Carr et al. (Chen, K. F., et al., Down-regulation of phospho-Akt is a major molecular determinant of bortezomib-induced apoptosis in hepatocellular carcinoma cells. Cancer Res, 2008. 68(16): p. 6698-707), the syringe was connected to a fluid column about 120 cm high. Measurements were performed for 3 repeats for each construct and six constructs in a group were evaluated to obtain the mean value.

The macro-porosity and permeability could be controlled by manipulating the gel volume fraction and the thickness of the gel filaments. Fifty percent and 40% gel volume fraction constructs were prepared by adding 1× and 1.5× volume of 0.03% mTGase solution to the hydrogel filaments for final cross-linking respectively. Two different pore sizes of stainless steel mesh (250-μm and 180-μm) were use to prepare gel filaments of different thickness.

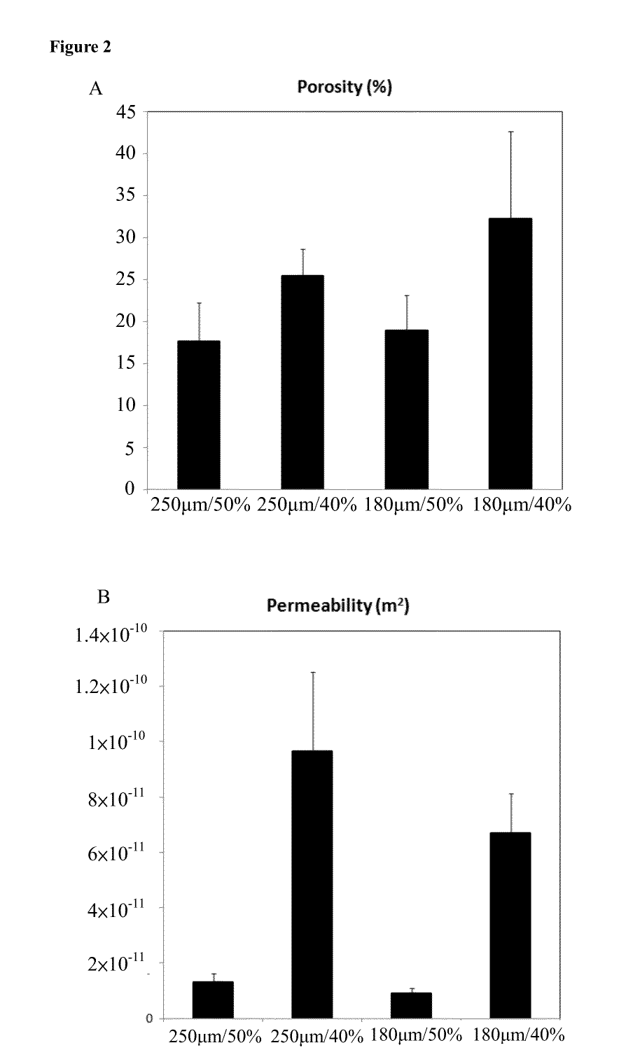

Theoretically, the porosity could be calculated as (1−[gel volume fraction]). However, the measured results were not consistent with the calculated value. A construct of 50% gel volume fracture has only about 20% measured porosity. Beside the errors of sampling and measurement, there might also be some variations in the volume of hydrogels after preparation, depending on the status of fluid content. Nevertheless, the measured porosity was directly related to the gel volume fraction, and not significantly related to the thickness of the filaments. The measured porosity with 50% and 40% gel volume fraction was (17.6±4.6)% and (25.4±3.2)% respectively for 250-μm mesh, and was (18.9±4.3)% and (32.2±10.5)% for 180-μm mesh (FIG. 2A).

The permeability was high enough for the constructs to be perfused with low pressure. Under the testing conditions (6 mL gel cylinder in a 10-mL syringe, under 120-cm high PBS column), it typically took only about 50 to 500 seconds to empty 10 mL of medium. The lower gel volume fraction and the thicker filament size resulted in higher permeability. The permeability with 50% and 40% gel volume fraction was (1.31±0.32)×10⁻¹¹ m² and (9.66±2.85)×10⁻¹¹ m² respectively for 250-μm mesh, and was (0.90±0.19)×10⁻¹¹ m² and (6.71±1.42)×10⁻¹¹ m² for 180-μm mesh (FIG. 2B).

### Example 3

### Cell Viability and Proliferation in the Constructs

For LIVE/DEAD stain, samples were transferred to a 24-well plate, washed with PBS, and 200 μL of staining solution containing 2 μM calcein AM (Life Technologies) and 4 μM Ethidium homodimer-1 (Eth-1; Life Technologies) in PBS was added. After incubated at 37° C. for 30 min, 2 μL of 100 μg/mL Hoechst 33342 (Sigma-Aldrich) was added, and the samples were examined by epifluorescence microscopy or confocal microscopy.

Hydrogels containing NIH 3T3 cells were fabricated as described. Static culture was performed by placing the constructs in 50-mL centrifuge tubes with 20 mL complete medium, changed every day. For perfusion culture, the constructs were situated in a 10-mL syringe connected within a closed loop perfusion system in a regular CO₂ incubator with filtered gas exchange. The medium reservoir contained 140 mL of complete medium, changed every 7 days. The perfusion rate of the medium was 1 mL/min. Six samples at each time point (at day 0 before put into 37° C. incubator, and at day 4 and 7) were evaluated for cell viability and proliferation.

The viability of the cells encapsulated in the constructs was examined by staining the samples with the LIVE/DEAD labeling reagents. The samples were examined using a 10× objective on an epifluorescence microscope. One hundred cells were assessed from three different areas of the same sample. The percentage of live cells was calculated.

Cell proliferation was estimated by counting cells after dissolving the gelatin constructs. Hydrogels were digested by 0.2% collagenase (Sigma-Aldrich) solution. Cells were pelleted and resuspended. Cell number was counted using a hematocytometer.

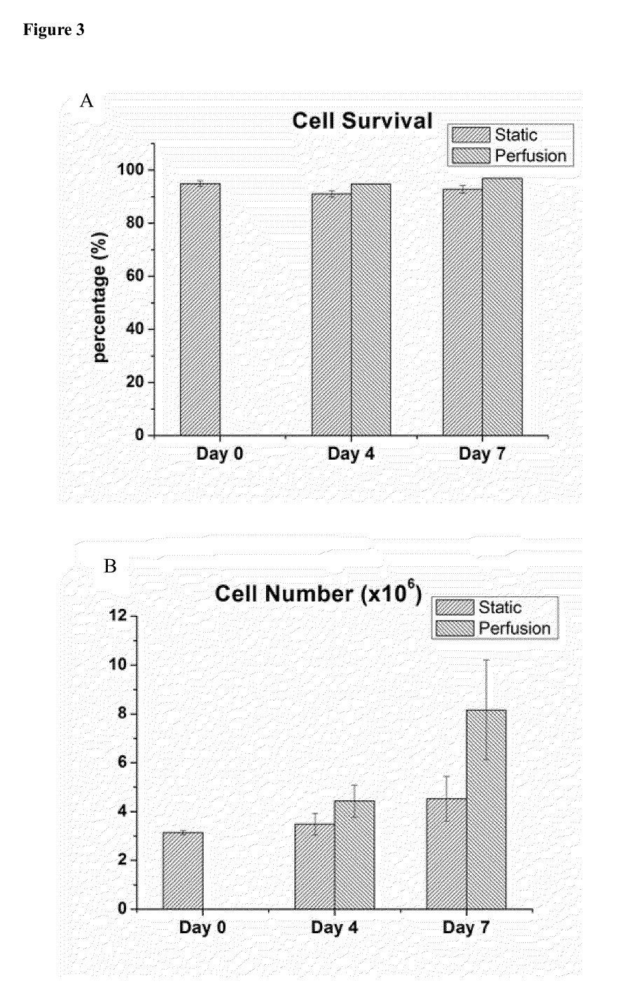

The viability of encapsulated cells was evaluated by manual counting of live versus total cells under epifluorescence microscopy. The survival rates of NIH 3T3 cells were uniformly high at day 0, 4, and 7. The fabrication process was well tolerated as indicated by the (94.9±1.1)% survival rate of the cells at day 0. The survival rate at day 4 and 7 was (91.1±1.2)% and (92.8±1.5)% respectively for static culture, and was (94.8±1.7)% and (96.9±0.9)% for perfusion culture (FIG. 3A).

The encapsulated cells could be retrieved by dissolving the gelatin hydrogel with collagenase, and the cell count was obtained. NIH 3T3 cells increased significantly at day 7, and the cell growth was significantly more under perfusion culture than under static culture. With initially (3.14±0.08)×10⁶ cells seeded in each construct at day 0, the cell count at day 4 and 7 was (3.48±0.45)×10⁶ and (4.52±0.92)×10⁶ respectively for static culture, and was (4.44±0.66)×10⁶ and (8.16±2.04)×10⁶ for perfusion culture (FIG. 3B).

### Example 4

### Cell Types and Growth Pattern

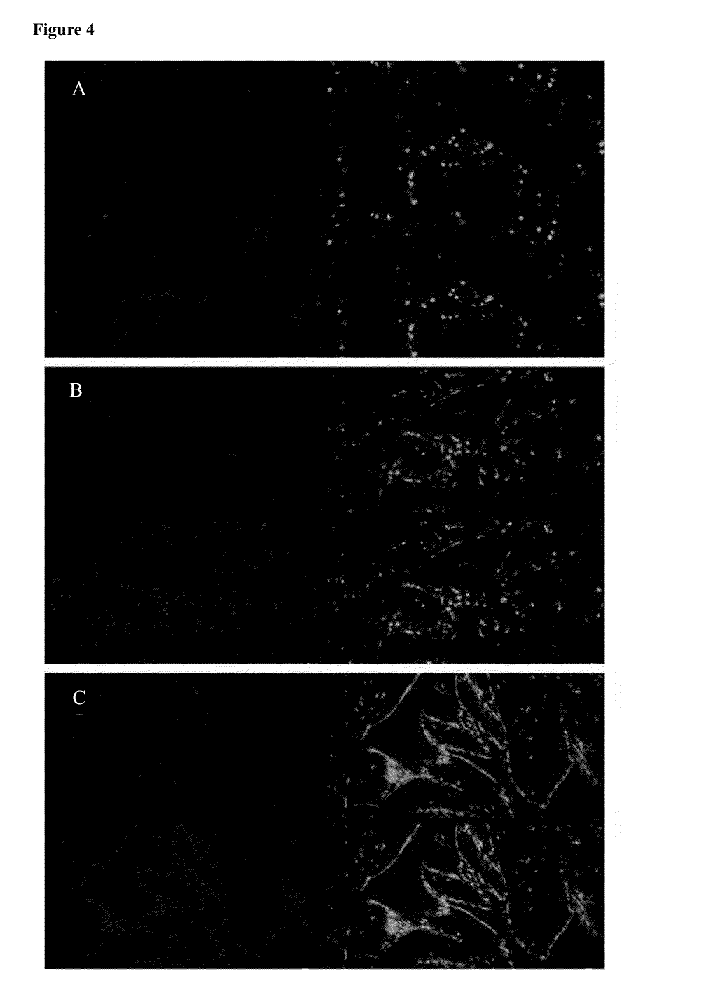

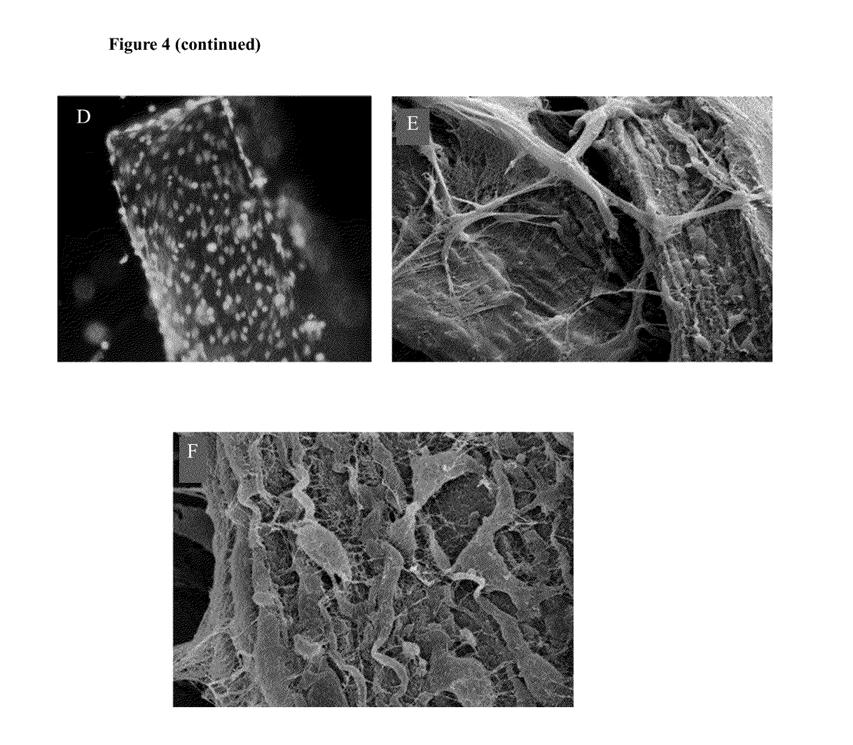

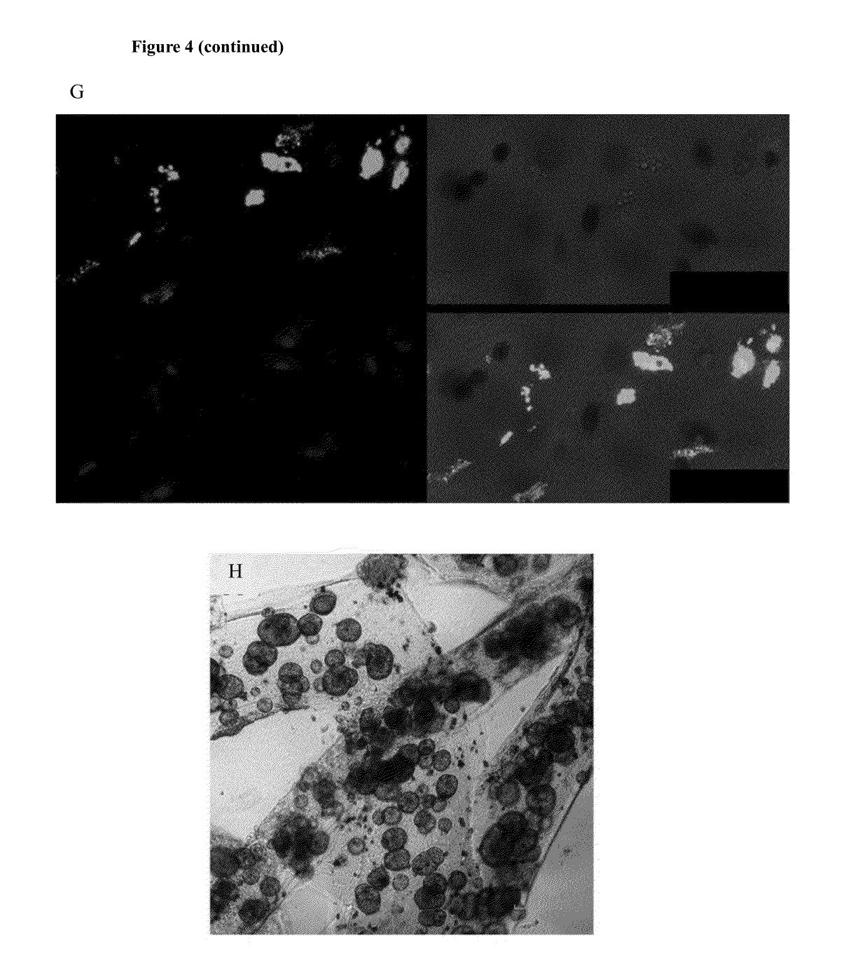

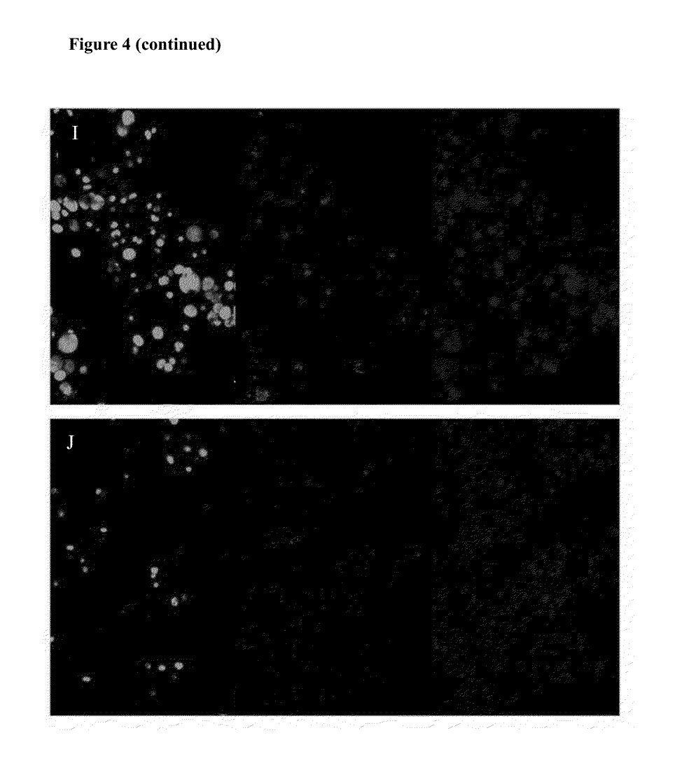

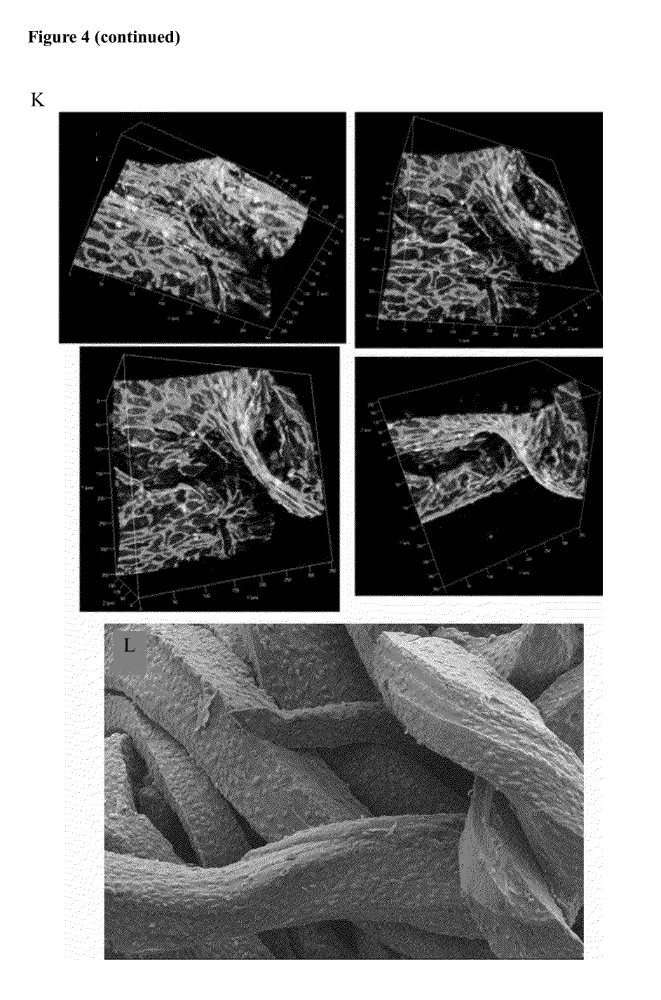

Different cell types showed different growth patterns in the scaffold. Under perfusion culture, the NIH 3T3 cells were initially round-shaped at day 0. Beside the increase in cell number, by the day 4, they were found to have out-stretched morphology in the hydrogel. At day 7, nearly all the NIH 3T3 cells had migrated out and attached on the surface of the hydrogel filaments (FIG. 4A-4C).

To recognize adipogenic differentiation from hADSCs, samples were incubated in a 24-well plate with 200 μL of 5 μM BODIPY 493/503 (Life Technologies) at room temperature for 30 min. Two microliters of 100 μg/mL Hoechst 33342/PBS was added before viewed under epifluorescence microscopy or confocal microscopy.

For adipogenic differentiation, constructs with encapsulated hADSCs were cultured in adipogenic medium containing DMEM, 10% FBS, 1 μM dexamethasone (Sigma-Aldrich), 0.1 mM indomethacin (Sigma-Aldrich), 0.5 mM 3-isobutyl-1-methylxanthine (IBMX; Sigma-Aldrich), 10 μM insulin (Sigma-Aldrich), and 100 U/mL penicillin-streptomycin for 3 weeks.

The growth pattern of hADSCs was similar to the NIH 3T3 cells. Human ADSCs migrated out and attached on the hydrogel surface (FIG. 4D). Under SEM, the cells showed out-stretched and flattened morphology, with active secretion of extracellular matrix (FIG. 4E & 4F). However, when the hADSCs were successfully induced into adipocytes, the cells remained inside the hydrogel (FIG. 4G).

The HuH-7 cells didn't have evident migration. After encapsulated in the hydrogel in the form of single-cell suspension, multiple-cellular spheres of about 100 μm in size could be obtained at day 7 of perfusion culture (FIG. 4H). HuH-7 cells also showed better proliferation under perfusion culture than under static culture (FIGS. 4I & 4J).

As evident from the experiments with NIH 3T3 cells and hADSCs, the constructs were also suitable for cell adhesion. The feasibility of seeding cells onto the surface of hydrogel filaments was examined. The HUVECs attached rapidly within 1 day, covered the surface evenly, and were able to sustain perfusion after 3 days (FIG. 4K & 4L).

### Example 4

### Bone Tissue Graft Engineering

For osteogenic differentiation, physically gelling gelatin hydrogel constructs with encapsulated hADSCs were cultured in osteogenic medium containing α-minimum essential medium (GibcoBRL; Grand Island, N.Y.) supplemented with 10% fetal bovine serum (HyClone, Logan, Utah), antibiotics (100 U/mL of penicillin G and streptomycin 100 μg/mL, GibcoBRL), ascorbic acid (50 mg/ml; Sigma Co, St. Louis, Mo.), and β-glycerophosphoric acid disodium salt (5 mmol/L; Sigma Co). For the visualization of mineralization, 10 m/mL tetracycline was supplemented in the culture medium.

Physically gelling gelatin hydrogel constructs induced into osteogenic grafts were removed from culture plates or perfusion vessels at 4-, 7-, 14, and 20-day. The fluid was evaporated in a dryer, and the constructs were immersed in 1 mL of 2N HCl overnight to dissolve the calcium salts. After neutralization with 2 mL of 1N NaOH, the calcium concentration was measured with Spectroquant Kit (Millipore, Darmstadt, Germany). FIG. 5 showed the gross appearance of osteogenic induction of hADSC in physically gelling gelatin hydrogel. Constructs with perfustion culture (lower row) showed more obvious opacification than that in static culture.

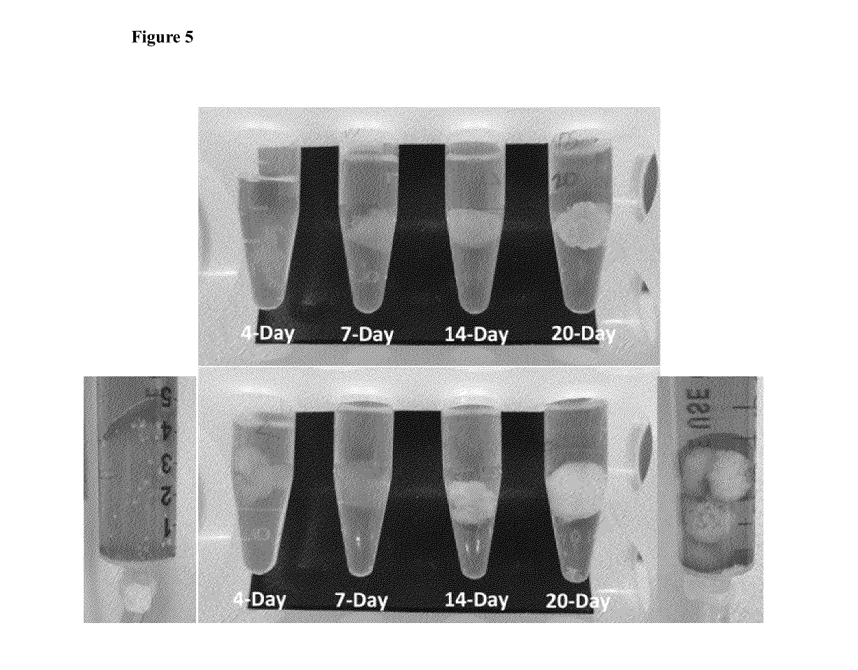

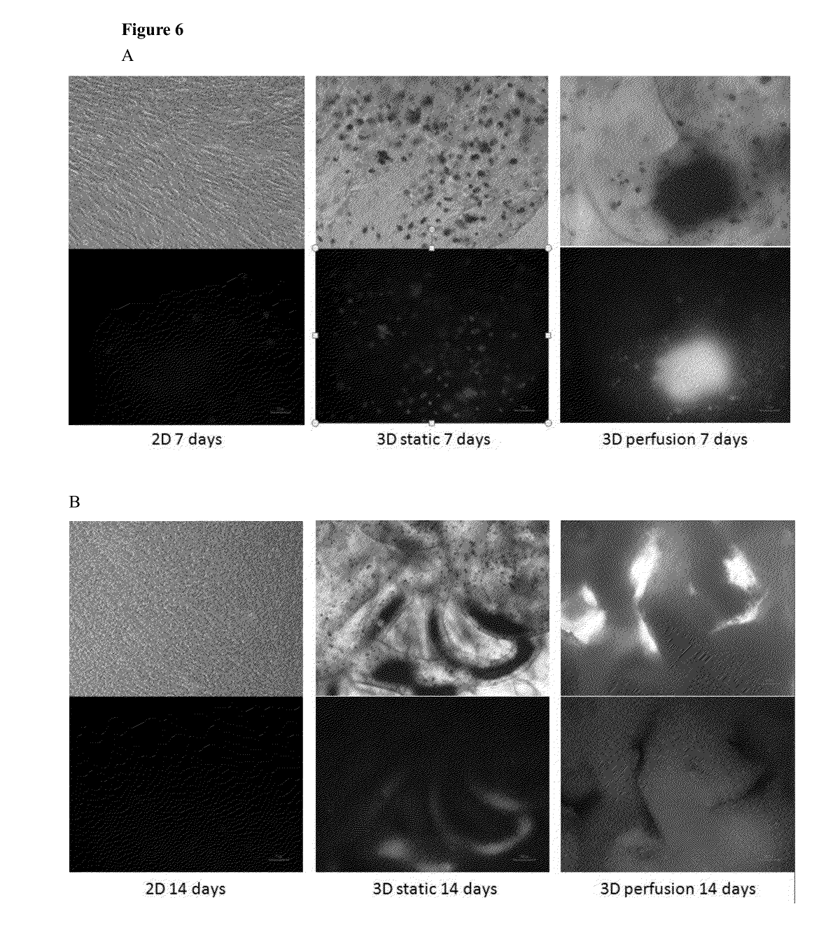

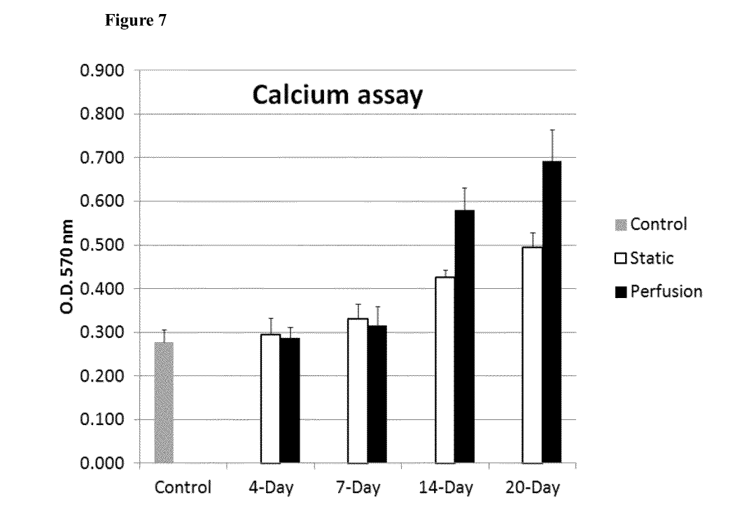

Osteogenic induction of hADSC encapsulated in the physically gelling gelatin hydrogel was striking. Once exposed to the osteogenic medium, hADSC could still express out-stretched morphology, but did not migrate out of the hydrogel vigorously. The mineralization activity as shown from tetracycline incorporation (FIG. 6A & 6B) and semi-quantitative calcium assay (FIG. 7) was higher in 3D environment than in 2D culture, and higher in perfusion culture than in static culture. The gelatin filaments showed evenly incorporated with tetracycline fluorescence by 14 days.

Constructs seeded with HUVECs were evaluated by immunofluorescent staining Mouse monoclonal anti-human CD31 antibody (R&D Systems, Minneapolis, Minn.) and rabbit polyclonal to α-smooth muscle actin antibody (Abcam, Cambridge, UK) were used as primary antibodies. The secondary antibodies were DyLight 488 Goat polyclonal to mouse antibody (Abcam) and pre-adsorbed PE Donkey F(ab′)2 polyclonal to rabbit antibody (Abcam). Samples were fixed with 4% paraformaldehyde (Sigma-Aldrich)/PBS for 10 min, permeabilized with 0.1% Triton ×100 (Sigma-Aldrich) in PBS for 10 min, and blocked with 3% BSA (Sigma-Aldrich)/PBS for 45 min. Samples were incubated with primary antibodies over night at 4° C., and then secondary antibodies (1:500 dilution) at room temperature for 1 hour. The nuclei were counter-stained by Hoechst 33342 in 1 μg/mL before observed under confocal microscopy.

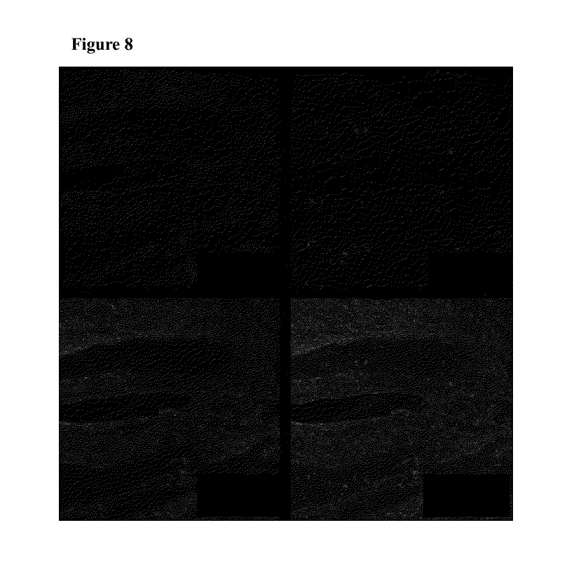

Osteogenesis and maintenance of endothelial growth needs different media and factors. The result showed that the two components could be implemented sequentially. Seeding of HUVEC after 1 week of osteogenic induction achieved concurrent visualization of the osseous component and even endothelial covering of the pore surface under confocal microscopy (FIG. 8).

What is claimed is:

1. A method for fabricating a cell-laden hydrogel construct, comprising the steps of:
(a) making a mixture of a physically gelable protein, a first crosslinking enzyme and a cell;
(b) extruding the mixture into a second crosslinking enzyme before the mixture is gelled and forming a microgel by physical gelling; and
(c) reacting the microgel with the second crosslinking enzyme to fabricate the cell-laden hydrogel construct.

2. The method of claim 1, wherein the concentration of the physically gelable protein in the mixture is from 24% to 1% w/v.

3. The method of claim 1, wherein the concentration of the first crosslinking enzyme in the mixture is from 0.6% to 0.01% w/v.

4. The method of claim 1, wherein the concentration of the second crosslinking enzyme is from 0.6% to 0.01% w/v.

5. The method of claim 1, wherein the temperature of the second crosslinking enzyme is from 30° C. to 0° C.

6. The method of claim 1, wherein the diameter of the microgel is from 400 μm to 100 μm.

7. The method of claim 1, wherein the physically gelable protein is gelatin.

8. The method of claim 1, wherein the first crosslinking enzyme is transglutaminase.

9. The method of claim 8, wherein the transglutaminase is microbial transglutaminase.

10. The method of claim 1, wherein the second crosslinking enzyme is transglutaminase.

11. The method of claim 10, wherein the transglutaminase is microbial transglutaminase.

12. The method of claim 1, wherein the extruding is through a syringe having a needle.

13. The method of claim 1, wherein the cell is a stem cell.

14. The method of claim 13, wherein the stem cell is a mesenchymal stem cell.

15. The method of claim 1, which is used for 3D cell culture.
